(19) United States
(12) Patent Application Publication
(10) Pub. No.: US 2022/0370757 A1
(43) Pub. Date: Nov. 24, 2022

(54) PERSONALIZED SLEEP WELLNESS SCORE FOR TREATMENT AND/OR EVALUATION OF SLEEP CONDITIONS

(71) Applicant: HypnoCore Ltd., Petach-Tikva (IL)

(72) Inventors: Yuval Eliezer ALTMAN, Tzur Yitzhak (IL); Shulamit EYAL, Givat Shmuel (IL); Armanda Lia BAHARAV, Tel-Aviv (IL)

(73) Assignee: HypnoCore Ltd., Petach-Tikva (IL)

(21) Appl. No.: 17/322,971

(22) Filed: May 18, 2021

(51) Int. Cl. A61M 21/02; G06N 20/10; G16H 50/30

(52) U.S. Cl. CPC A61M 21/02; G06N 20/10; G16H 50/30; A61M 2021/0044

(57) ABSTRACT

There is provided a method of training a machine learning model for generating a sleep wellness score used for treatment of a sleep condition in a target individual, comprising: providing a baseline machine learning model with weights set to initial baseline values, accessing sleep-parameters computed for historical sleep sessions of the target individual, training the baseline machine learning model using the sleep-parameters for the historical sleep sessions of the target individual by adjusting the initial baseline values of the weights, to obtain a customized machine learning model, accessing sleep-parameters computed for previous sleep session(s) of the target individual, inputting the sleep-parameters computed for previous sleep session(s) into the customized machine learning model, and obtaining a sleep wellness score as an outcome of the customized machine learning model.

### FIELD AND BACKGROUND OF THE INVENTION

The present invention, in some embodiments thereof, relates to treatment of sleep conditions and, more specifically, but not exclusively, to systems and methods for computing a sleep wellness score for treatment and/or evaluation of sleep conditions.

Sleep is a highly complex physiological and psychological state. Lack of sufficient amount of sleep, and/or lack of sufficient quality sleep, may cause a person to feel tired the next day, reducing the person's ability to focus, and/or generally function in a productive state.

Different sleep improvement approaches have been developed to try and improve the amount and/or quality of sleep the person receives.

### SUMMARY OF THE INVENTION

According to a first aspect, a computer implemented method of training a machine learning model for generating a sleep wellness score used for treatment of a sleep condition in a target individual, comprises: providing a baseline machine learning model with a plurality of weights set to initial baseline values, accessing a plurality of sleep-parameters computed for a plurality of historical sleep sessions of the target individual, training the baseline machine learning model using the plurality of sleep-parameters for the plurality of historical sleep sessions of the target individual by adjusting the initial baseline values of the plurality of weights, to obtain a customized machine learning model, accessing a plurality of sleep-parameters computed for at least one previous sleep session of the target individual, inputting the plurality of sleep-parameters computed for at least one previous sleep session into the customized machine learning model, and obtaining a sleep wellness score as an outcome of the customized machine learning model.

According to a second aspect, a computer implemented method of using a machine learning model for generating a sleep wellness score used for treatment of a sleep condition in a target individual, comprises: accessing a plurality of sleep-parameters computed for at least one previous sleep session of the target individual, inputting the plurality of sleep-parameters computed for at least one previous sleep session into a customized machine learning model, and obtaining a sleep wellness score as an outcome of the customized machine learning model, wherein the customized machine learning model is trained by: providing a baseline machine learning model with a plurality of weights set to initial baseline values, accessing a plurality of sleep-parameters computed for a plurality of historical sleep sessions of the target individual, and training the baseline machine learning model using the plurality of sleep-parameters for the plurality of historical sleep sessions of the target individual by adjusting the initial baseline values of the plurality of weights, to obtain a customized machine learning model.

According to a third aspect, a system for training a machine learning model for generating a sleep wellness score used for treatment of a sleep condition in a target individual, comprises: at least one hardware processing executing a code for: accessing a baseline machine learning model with a plurality of weights set to initial baseline values, accessing a plurality of sleep-parameters computed for a plurality of historical sleep sessions of the target individual, training the baseline machine learning model using the plurality of sleep-parameters for the plurality of historical sleep sessions of the target individual by adjusting the initial baseline values of the plurality of weights, to obtain a customized machine learning model, accessing a plurality of sleep-parameters computed for at least one previous sleep session of the target individual, inputting the plurality of sleep-parameters computed for at least one previous sleep session into the customized machine learning model, and obtaining a sleep wellness score as an outcome of the customized machine learning model.

In a further implementation form of the first, second, and third aspects, further comprising analyzing the sleep wellness score to identify the sleep condition by feeding the sleep wellness score into an application selected from a group consisting of: a sleep evaluation application, a sleep improvement application, a sleep monitoring application, a sleep maintenance application, and presenting instructions on a display and/or for playing on speakers for treating the target user for the sleep condition and/or for gaining insights into the sleep condition according to the analysis.

In a further implementation form of the first, second, and third aspects, wherein the baseline machine learning model comprises a previous version of the customized machine learning model previously trained on sleep-parameters for historical sleep session, and the customized machine learning model comprises a current version thereof trained on sleep-parameters of most recent sleep session that is later than the historical sleep session, and further comprising: iterating over a plurality of sequential time intervals, dynamically re-training the current version of the customized machine learning model using the plurality of sleep-parameters for most recent historical sleep session by adjusting previously computed values of the plurality of weights of previous versions of the customized machine learning model, wherein the accessing comprises accessing the plurality of sleep-parameter for the most recent previous sleep session, and iterating the inputting, and the obtaining over the plurality of sequential time intervals to obtain a respective sleep wellness score for each most recent previous sleep session.

In a further implementation form of the first, second, and third aspects, wherein dynamically re-training comprises re-training the customized machine learning model using the plurality of sleep-parameters for the most recent historical sleep session labelled with the sleep wellness score obtained as an outcome of the previous version of the customized machine learning model, wherein a current version of the customized machine learning model is further fed an input of at least one historical sleep wellness score with respective plurality of sleep-parameter for the most recent previous sleep session.

In a further implementation form of the first, second, and third aspects, further comprising analyzing a plurality of sleep wellness scores obtained over the plurality of sequential time intervals to detect a statistically significant deviation of a certain sleep wellness score, and identifying at least one sleep-parameter most significantly contributing to the certain sleep wellness score outcome by the customized machine learning model.

In a further implementation form of the first, second, and third aspects, further comprising: at least one of: (i) reducing previously computed values of a first sub-set of the plurality of weights associated with a first sub-set of sleep-parameters that are statistically constant over a plurality of sleep sessions, and (ii) increasing previously computed values of a second sub-set of the plurality of weights associated with a second sub-set of sleep-parameters that are statistically varying over the plurality of sleep sessions.

In a further implementation form of the first, second, and third aspects, the baseline machine learning model and the customized machine learning model are implemented as an auto-regressive model.

In a further implementation form of the first, second, and third aspects, the initial baseline values of the plurality of weights are initially set to random values.

In a further implementation form of the first, second, and third aspects, the baseline machine learning model is trained on a training dataset that includes a plurality of sample sleep-parameters labelled with respective sample sleep wellness scores denoting ground truth for a plurality of sample individuals, wherein the training the baseline machine learning model to obtain the customized machine learning model is done on a customized training dataset that includes the plurality of sleep-parameters of the target individual and excludes sleep-parameters of other individuals.

In a further implementation form of the first, second, and third aspects, further comprising extracting a plurality of features from the plurality of sleep-parameters, wherein the plurality of features are used to train the baseline machine learning model and/or are fed into the customized machine learning model.

In a further implementation form of the first, second, and third aspects, the plurality of features are customized by being selected according to a set of characteristics of the target user denoting the target user's sleep behavior and/or history and/or demographic parameters of the target user.

In a further implementation form of the first, second, and third aspects, further comprising creating a historical feature dataset that maps each respective historical sleep session to a respective set of feature extracted from sleep-parameters obtained from the respective historical sleep session, wherein the historical feature dataset excludes features extracted from sleep-parameters of the at least one previous sleep session.

In a further implementation form of the first, second, and third aspects, further comprising: performing a principal component analysis (PCA) of the historical feature dataset by applying an alternating least squares (ALS) process and weighting observations with a temporal function indicating time from observation, to obtain a principal component coefficient dataset and a vector documenting percentage of total variance explained by each principal component, computing a weight dataset as a weighted average of the principal component coefficient matrix, with respect to values of the vector, wherein baseline machine learning model with weights set to initial baseline values is implemented as the weight dataset.

In a further implementation form of the first, second, and third aspects, further comprising: normalize weights of the weight dataset within a defined range, adjust sign values of each weight in the weight dataset based on a predefined directions vector, and proportionally distribute weights of missing features in the weight dataset to available feature weights in the weight dataset.

In a further implementation form of the first, second, and third aspects, wherein baseline machine learning model with weights set to initial baseline values is implemented as the weight dataset, wherein training the baseline machine learning model to obtain the customized machine learning model comprises a recent feature dataset of features extracted from sleep-parameters of the at least one previous sleep session, wherein obtaining the sleep wellness score as the outcome of the customized machine learning model comprises computing the sleep wellness score as a weighted sum of the recent feature dataset, with respect to the weight dataset.

In a further implementation form of the first, second, and third aspects, further comprising heuristically correcting the sleep wellness score based on predefined discrepancies between the sleep wellness score and values of predefined features.

In a further implementation form of the first, second, and third aspects, further comprising computing at least one feature having largest negative effect on the sleep wellness score, by: grouping the features of the historical feature dataset into a plurality of feature groups, computing a weighted contribution of respective features of each respective feature group on the sleep wellness score based on the weight dataset and the recent feature dataset, selecting at least one feature group which had a largest negative contribution to the sleep wellness score with absolute values larger than a threshold.

In a further implementation form of the first, second, and third aspects, the sleep wellness score is represented as a numerical value, and further comprising classifying the sleep wellness score into one of a plurality of classification categories based on predefined thresholds.

In a further implementation form of the first, second, and third aspects, further comprising computing a reliability level for the calculation of the sleep wellness score based on at least one of: (i) an amount of data missing from the recent feature dataset, (ii) a number of features in the historical feature dataset, and (iii) amount of data missing from the historical feature dataset.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of components of a system 100 for dynamically training a machine learning model and using the machine learning model for generating a sleep wellness score for treatment of a sleep condition in a target individual, in accordance with some embodiments of the present invention;

FIG. 2 is a flowchart of a method of dynamically training a machine learning model and using the machine learning model for generating a sleep wellness score for treatment of a sleep condition in a target individual, in accordance with some embodiments of the present invention; and

FIG. 3 is a flowchart of another exemplary method for computing a sleep wellness score for a user, in accordance with some embodiments of the present invention.

### DESCRIPTION OF SPECIFIC EMBODIMENTS OF THE INVENTION

The present invention, in some embodiments thereof, relates to treatment of sleep conditions and, more specifically, but not exclusively, to systems and methods for computing a sleep wellness score for treatment and/or evaluation of sleep conditions.

As used herein, a sleep condition may be a clinically diagnosed sleep disorder, for example, insomnia. The sleep condition may relate to one or more of the following: The sleep condition may be a subject state in individuals which are not suffering from a clinically diagnosed sleep disorder. The sleep condition may be in individuals that may or may not have a history of sleep disorders, and/or may or may not currently have a healthy sleep. The sleep condition may relate to a state of sleep, for which improvement may be desired. The sleep condition may relate to a state of sleep, for which additional insight is desired. The sleep condition may relate to a state of sleep, which may be good sleep, which the user desires to maintain. The sleep condition may be in individuals that have poor sleep hygiene and/or sleep-related habits, which may or may not impair their sleep quality and/or duration, for example, inconsistent wake up times, caffeine consumption before bedtime, alcohol consumption before bedtime, exercising prior to bedtime, suboptimal bed environment in terms of noise, temperature, light, etc. The sleep condition may be secondary related to a primary condition, optionally medical condition, for which the sleep condition is acting as a secondary condition, for example, due to pain, arthritis, cardiovascular condition, hypertension, diabetes, lung disease, allergies, depression, anxiety, post-traumatic stress disorder, or other conditions. It is noted that that the sleep wellness score described herein does not necessarily aim to aid in treating these primary conditions directly, but may aid in improvement of the secondary sleep condition, such as for any sleep-related treatment, evaluation, maintenance, monitoring etc.

An aspect of some embodiments of the present invention relates to systems, methods, a computing device, and/or code instructions (stored on a memory and executable by hardware processors) for dynamically training a machine learning model to generate a customized machine learning model that generates an outcome of a sleep wellness score, where the analysis of the sleep wellness score is used to evaluate and/or treat a sleep condition in a target individual (also referred to herein as a user). A baseline machine learning model (e.g., auto-regressor) with a set of initial weights is provided. During training iterations, the baseline machine learning model may be a previous version of the customized machine learning model. The training dataset used to iteratively train the baseline and/or previous versions of the machine learning model include historical data of the same target user for which the trained and/or updated machine learning model is used for inference, optionally including only historical data of the same target user and/or excluding data of other users that are not the target user. The baseline machine learning model is trained using sleep-parameters computed for multiple historical sleep sessions of the target individual, by adjusting the initial weights, to obtain a customized machine learning model. Sleep-parameters computed for previous sleep session(s), which may be more recent than sleep-parameters of the historical sleep session used to train the baseline machine learning model, are fed into the customized machine learning model. A sleep wellness score is obtained as an outcome of the customized machine learning model.

Alternatively or additionally, the baseline and/or previous versions of the machine learning model may include sleep-parameters and/or other data from other sample subjects. The subjects may be selected, for example, based on having similar and/or same attributes as a target user, for example, similar sleep-parameters, similar demographic parameters, and/or similar behavior.

The sleep wellness score may represent a personalized score for the target individual, indicating a state of a sleep condition of the target individual. The sleep wellness score may be analyzed, for example, to identify the sleep condition and/or evaluate the sleep condition, in a personalized manner. The customized machine learning model is dynamically updated over time using new sleep-parameters, which may enable the customized machine learning model to “learn” the sleep-parameters of the target individual and their impact on the sleep wellness score. For example, the customized machine learning model learns the baseline sleep-parameters and the baseline of the sleep wellness score. This may enable detecting significant deviations from the baseline of the sleep wellness score. A trend in sleep wellness scores over time may be analyzed, for example, to determine whether current sleep wellness score statistically significantly deviate from the trend, such as higher scores (e.g., indicating improvement in the sleep condition during that sleep session) and/or lower scores (e.g., indicating deterioration in the sleep condition during that sleep session). For example, changes in the value of the sleep wellness score may indicate changes in the sleep condition, which may indicate improved sleep (e.g., sleep wellness score increased over a past baseline). In another example, learning over time which sleep-parameters are constant and reducing their impact on the sleep wellness score, and/or learning over time which sleep-parameters are dynamically changing and increasing their impact on the sleep wellness score.

An analysis (e.g., machine learning interpretability analysis) of the impact of the sleep-parameters on the computation of the sleep wellness score by the customized machine learning model may be done to identify the sleep-parameter(s) with greatest impact, which may be positive and/or negative. Instructions for changing (e.g., when negative) and/or for maintaining (e.g., when positive) the sleep-parameters may be generated, for example, too many coffees per day may be identified as significantly impacting poor sleep—indicating to the user that reducing the coffees will be greatly beneficial to improve sleep. Exercise during the day may be identified as significantly providing restful sleep—indicating to the user to continue the exercise to maintain the restful sleep.

The customized machine learning model “learns” the sleep-parameters and related sleep wellness score of the target individual. For example, a first person may sleep 6 hours a night, work a stressful job, and drink many coffees, however, the first person may sleep well and wakes up fresh and has a productive day. Now, a second person may have a similar profile, sleeping 6 hours a night, working a stressful job, and drinking many coffees, but the second person may sleep poorly, wake up exhausted, and have an unproductive day. The customized machine learning model “learns” the sleep-parameter for the first person, and generates the sleep wellness score accordingly, indicating, for example, a good sleep condition. Another customized machine learning model “learns” the sleep-parameter for the second person, which are noted to be mostly similar to the sleep-parameters of the first person, and generates a different sleep wellness score, indicating, for example, a poor sleep condition. This enables providing different treatment to the first and second individuals. For example, the first individual may be encouraged to continue as-is, and/or to make small improvements. The second individual may be identified as having a problem which should be addressed immediately with drastic measures. It is noted that the customized machine learning model that “learns” the sleep-parameters of each individual enables customized approaches, which is in contrast to other approaches, such as using a general model for all individual. Such general model would compute the same score for the two people, and be unable to capture the personalized differences between them, even for similar sleep-parameters. Customized treatment cannot be provided by such general model, but customized treatment may be provided using the customized machine learning model described herein.

At least some implementations of the systems, methods, apparatus, and/or code instructions described herein address the medical problem of treating a target individual suffering from a sleep condition. Sleep is a highly complex physiological and psychological state. Lack of sufficient amount of sleep, and/or lack of sufficient quality sleep, may cause a person to feel tired the next day, reducing the person's ability to focus, and/or generally function in a productive state. The challenge is that sleep may vary from night to night, making it difficult to dynamically identify a changing sleep condition and to dynamically adapt the treatment for the sleep condition.

At least some implementations of the systems, methods, apparatus, and/or code instructions described herein improve the medical field of sleep conditions, by providing a personalized sleep wellness score which enables identifying the sleep condition the target individual, selecting which sleep application to use, tracking the sleep condition over multiple sleep session, and selecting treatment for the sleep condition, while dynamically adjusting to changes in sleep sessions and/or other characteristics of the target user.

At least some implementations of the systems, methods, apparatus, and/or code instructions described herein address the technical problem of computing an objective, reproducible metric, indicative of quality of sleep of an individual. Generally, the perception of sleep quality and quantity, as well as sleep needs, are highly individual. Hence, it is challenging to develop a single metric which can evaluate the sleep of different people. Moreover, a person's sleep behavior may change over time, due to multiple reasons, e.g., a lifestyle change, workplace change, change in familial status, change in health condition etc.

At least some implementations of the systems, methods, apparatus, and/or code instructions described herein improve the technical fields of sleep improvement applications, by providing an objective, reproducible metric, used to evaluate sleep of an individual. The metric described herein is personalized for each individual and/or adaptive to the individual. In some implementations, the improvement is provided by an auto-regressive machine learning model that learns from the user's own sleep data, and uses that user's own sleep data to compute a personalized and/or adaptive sleep wellness score for the specific user. As different individuals differ from one another, the model is designed to adapt based on the individual user's data. In some implementations, a personalized variability-based machine learning model computes the personalized and/or adaptive sleep wellness score.

The machine learning model described herein learns from the user's own history of sleep parameters, to compute the personalized and/or adaptive sleep wellness score. Features extracted from the sleep parameters may be customized and/or selected according to user parameters of the specific user, adding another layer of customization. Weights used by the machine learning model to compute the sleep wellness score are personalized and adapted to the specific user by being set according to the history of sleep parameters and/or according to customized features extracted from the history of sleep parameters of the specific user. This creates a customized machine learning model that is specifically trained for each user.

In other implementations described herein, a more generic machine learning model is generated by being trained on sleep parameters and/or other parameters of different people. The generic model may be trained on a training dataset from different people, or may be trained on a specific training dataset of people selected by having common sleep parameters and/or other common attributes such as behavior and/or demographics. Different generic models may be trained for different groups of people with different attributes, for example, a model for overnight shift workers, another model for people suffering from a clinical sleep disorder, and yet another model for those trying to maintain current sleep conditions. Such more generic machine learning model may use weights which are determined based on data from many different sample individuals. The same generic machine learning model may then be used for computing scores for many different target users. The target user may select the generic model trained on a training dataset of data taken from subjects with attributes similar to the target user. The generic machine learning model may use a predefined set of parameters, with a predefined set of weights, for all people, to generate some sort of a score for a person's sleep, which may disregard adaptiveness, and personalization. The generic model may then be used as the baseline model, as described herein, which may then be further personalized for each target user, as described herein.

At least some implementations use the customized machine learning model that adapts over time. For example, an event caused a change in a person's sleep behavior, such as a new workplace, the birth of a new child, a change in health condition, etc. The customized machine learning model described herein detect the change in sleep behavior of the specific user, and/or adapts the model's knowledge of the specific user's sleep baselines based on the new conditions.

At least some implementations personalize adaptation of the weights of the customized machine learning model. For example, a person has a consistent sleep schedule, with constant bedtime and wake up times. Weights assigned to constant parameters do not contribute a lot of information for most of the time, since these parameters are less likely to change over time. At least some implementations provide the improvement assigning relatively smaller weights for these parameters, as the possible gain in information they provide regarding the person's changes in sleep patterns, is presumably smaller, allowing to assign relatively larger weights for other parameters with larger potential information gain.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

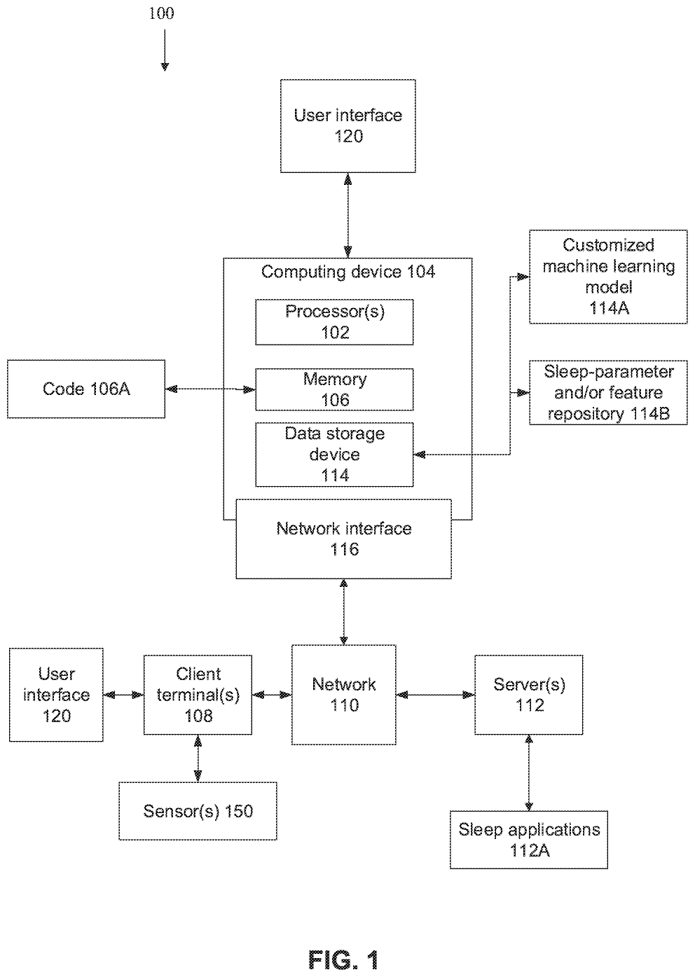

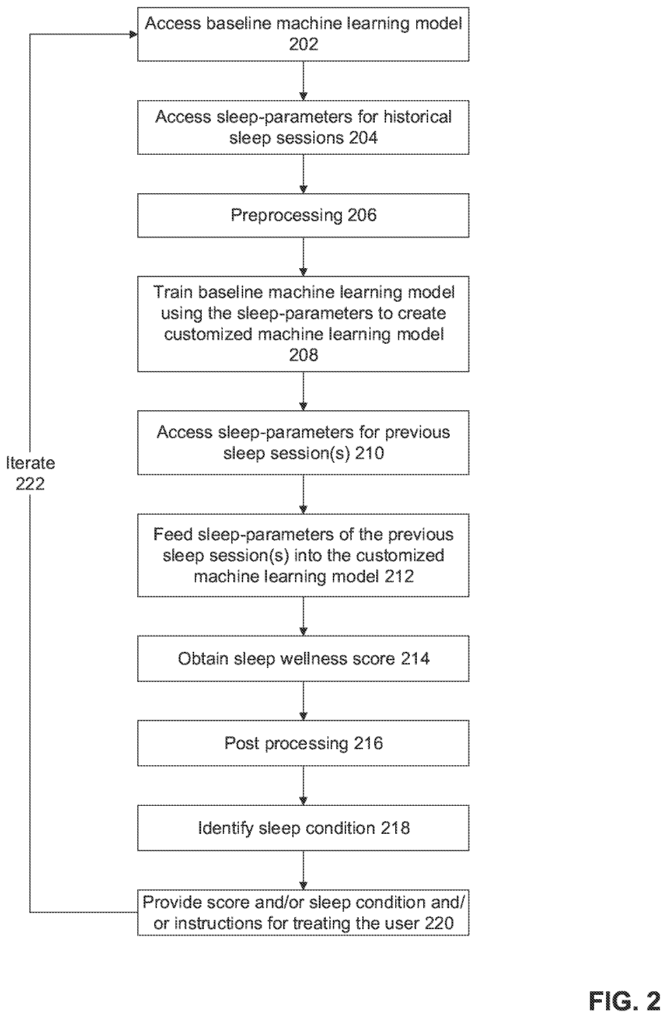

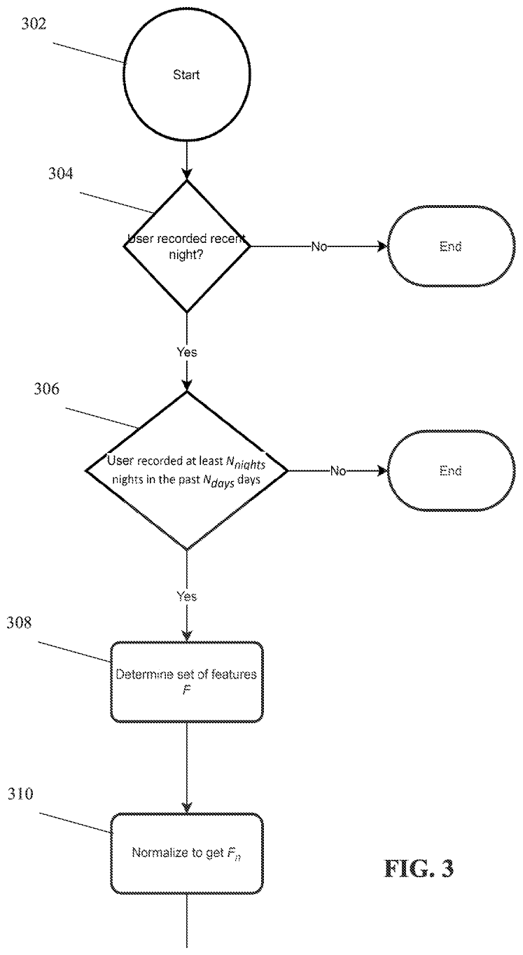

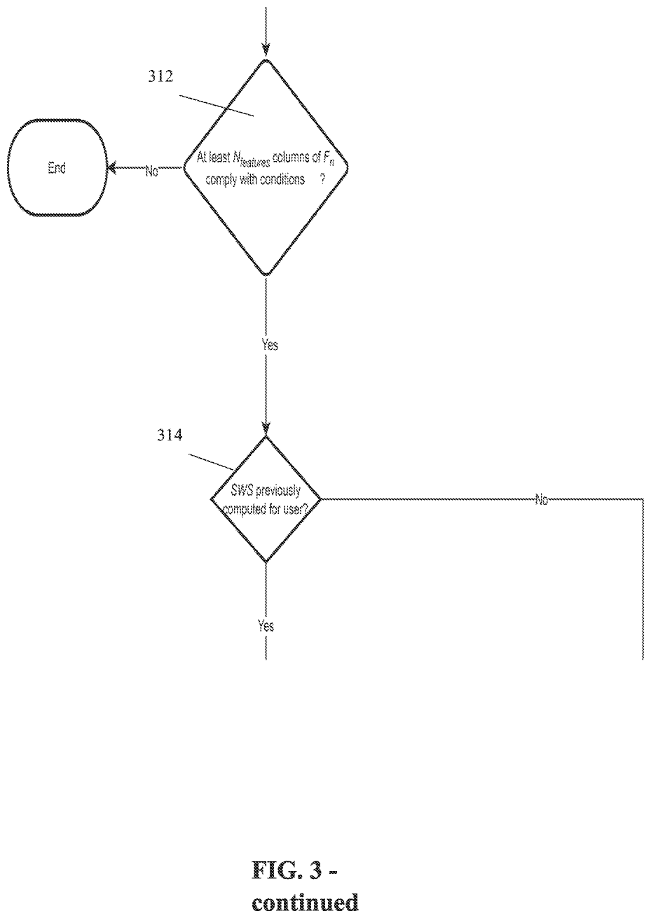

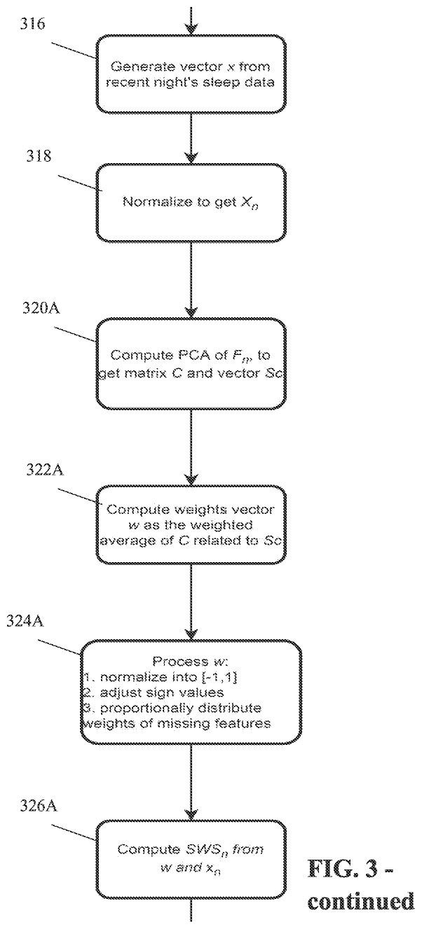

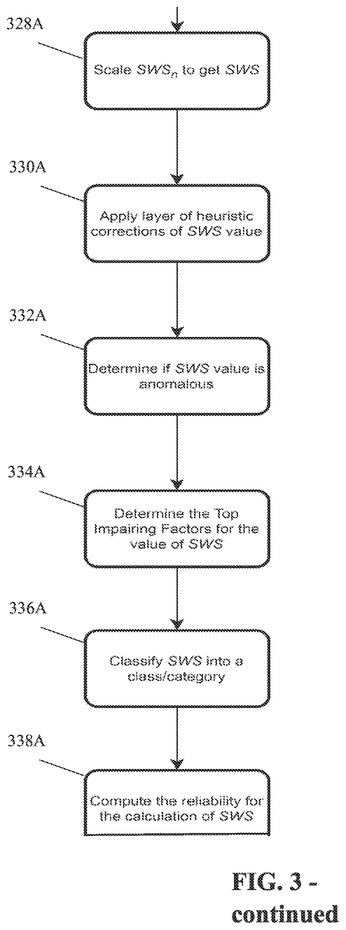

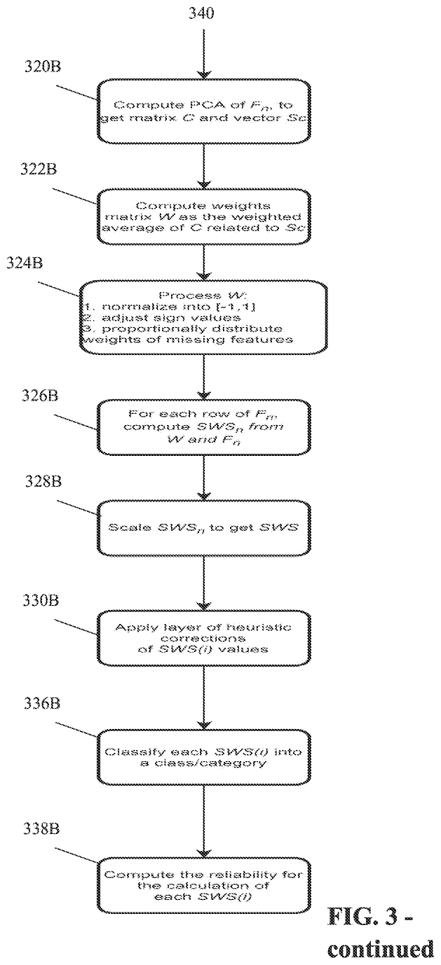

Reference is also made to FIG. 1, which is a block diagram of components of a system 100 for dynamically training a machine learning model and using the machine learning model for generating a sleep wellness score for treatment of a sleep condition in a target individual, in accordance with some embodiments of the present invention. Reference is also made to FIG. 2, which is a flowchart of a method of dynamically training a machine learning model and using the machine learning model for generating a sleep wellness score for treatment of a sleep condition in a target individual, in accordance with some embodiments of the present invention. Reference is also made to FIG. 3, which is a flowchart of another exemplary method for computing a sleep wellness score for a user, in accordance with some embodiments of the present invention. The method of FIG. 3 may be combined with and/or based on the method described with reference to FIG. 2. For example, the method described with reference to FIG. 2 may be a higher level description, with the method described with reference to FIG. 3 providing additional details and/or additional features of an exemplary lower level approach. System 100 may implement the acts of the method described with reference to FIGS. 2-3, by processor(s) 102 of a computing device 104 executing code instructions stored in a memory 106 (also referred to as a program store).

Computing device 104 may be implemented as, for example one or more and/or combination of: a group of connected devices, a client terminal, a server, a virtual server, a computing cloud, a virtual machine, a desktop computer, a thin client, a network node, and/or a mobile device (e.g., a Smartphone, a Tablet computer, a laptop computer, a wearable computer, glasses computer, and a watch computer).

Multiple architectures of system 100 based on computing device 104 may be implemented. For example:
Computing device 104 may be implemented as a standalone device (e.g., kiosk, client terminal, smartphone) that include locally stored code instructions 106A that implement one or more of the acts described with reference to FIGS. 2-3. The locally stored instructions may be obtained from another server, for example, by downloading the code over the network, and/or loading the code from a portable storage device. For example, sleep-parameters (and/or data for computing the sleep-parameters) are locally entered by a user using client terminal 104 and/or collected by sensors connected to computing device 104. Computing device locally trains a customized machine learning model for the specific user, and provides the sleep wellness score for the specific user. The respective sleep wellness score and/or respective instructions to improve sleep determined according to the respective sleep wellness score may be provided to the specific user, for example, presented on a display of computing device 104.
Computing device 104 executing stored code instructions 106A, may be implemented as one or more servers (e.g., network server, web server, a computing cloud, a virtual server) that provides centralized services (e.g., one or more of the acts described with reference to FIGS. 2-3) to one or more client terminals 108 over a network 110. For example, providing software as a service (SaaS) to the client terminal(s) 108, providing software services accessible using a software interface (e.g., application programming interface (API), software development kit (SDK)), providing an application for local download to the client terminal(s) 108, providing an add-on to a web browser running on client terminal(s) 108, and/or providing functions using a remote access session to the client terminals 108, such as through a web browser executed by client terminal 108 accessing a web sited hosted by computing device 104. For example, sleep-parameters (and/or data for computing the sleep-parameters) are provided from each respective client terminal 108 of each respective user to computing device 104. Computing device centrally trains a respective customized machine learning model for each respective user, and provides a respective sleep wellness score for each specific user. The respective sleep wellness score and/or respective instructions to improve sleep determined according to the respective sleep wellness score may be provided to each respective client terminal 108, for each specific user.

Hardware processor(s) 102 of computing device 104 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 102 may include a single processor, or multiple processors (homogenous or heterogeneous) arranged for parallel processing, as clusters and/or as one or more multi core processing devices.

Memory 106 stores code instructions executable by hardware processor(s) 102, for example, a random access memory (RAM), read-only memory (ROM), and/or a storage device, for example, non-volatile memory, magnetic media, semiconductor memory devices, hard drive, removable storage, and optical media (e.g., DVD, CD-ROM). Memory 106 stores code 106A that implements one or more features and/or acts of the method described with reference to FIGS. 2-3 when executed by hardware processor(s) 102.

Computing device 104 may include a data storage device 114 for storing data, for example, the customized machine learning model 114A which is dynamically updated as described herein, and/or a sleep-parameter and/or feature (which are extracted from the sleep-parameters) repository 114B. Data storage device 114 may be implemented as, for example, a memory, a local hard-drive, virtual storage, a removable storage unit, an optical disk, a storage device, and/or as a remote server and/or computing cloud (e.g., accessed using a network connection).

Network 110 may be implemented as, for example, the internet, a local area network, a virtual network, a wireless network, a cellular network, a local bus, a point to point link (e.g., wired), and/or combinations of the aforementioned.

Computing device 104 and/or client terminal(s) 108 may be in communication with one or more sensor(s) 150 that perform measurements for collecting sleep-parameters, as described herein in additional detail.

Computing device 104 may include a network interface 116 for connecting to network 110, for example, one or more of, a network interface card, a wireless interface to connect to a wireless network, a physical interface for connecting to a cable for network connectivity, a virtual interface implemented in software, network communication software providing higher layers of network connectivity, and/or other implementations.

It is noted that in the standalone implementation, network interface 116 is not necessarily required, as computing device 104 includes sensors 150 and/or user interface 120 in a single device that may operate without externally communication with other devices, for example, a smartphone, a kiosk, and a dedicated device.

Computing device 104 may connect using network 110 (or another communication channel, such as through a direct link (e.g., cable, wireless) and/or indirect link (e.g., via an intermediary computing unit such as a server, and/or via a storage device) with one or more of:
Remote server(s) 112 running one or more sleep applications 112A. The sleep wellness score computed by computing device 104 is provided as input into the sleep application(s) 112A, for example, for generating instructions to the user on how to improve sleep according to their sleep wellness score.
Client terminal(s) 108, when computing device 104 is implemented as a server remotely providing the features and/or acts described with reference to FIGS. 2-3.
Sensor(s) 150 that perform measurements for collecting sleep-parameters.

Computing device 104 and/or client terminal(s) 108 include and/or are in communication with one or more physical user interfaces 120 that include a mechanism for a user to enter data (e.g., manually enter sleep-parameters) and/or view the displayed results (e.g., computed sleep wellness score, instructions to improve sleep), within a GUI. Exemplary user interfaces 120 include, for example, one or more of, a touchscreen, a display, gesture activation devices, a keyboard, a mouse, and voice activated software using speakers and microphone.

Referring now back to FIG. 2, at 202, a baseline machine learning model is provided and/or accessed.

The baseline machine learning model may be set to an initial state. For example, weights of the baseline machine learning model are set to initial baseline values, for example, set to random values. Such initial setting may be provided during a first iteration, when the baseline machine learning model is first used. Alternatively, the baseline machine learning model is trained on a training dataset that includes sample sleep-parameters labelled with respective sample sleep wellness scores denoting ground truth for multiple sample individuals. In such implementation, the training of the baseline machine learning model to obtain the customized machine learning model, as described with reference to 208, may be done on a customized training dataset that includes sleep-parameters of the target individual and excludes sleep-parameters of other individuals.

It is noted that during iterations described herein, the baseline machine learning model is a previous version of the customized machine learning model which has been previously trained on sleep-parameters for historical sleep session, as descried with reference to 208.

At 204, sleep-parameters computed for multiple historical sleep sessions of the target individual are accessed.

The sleep-parameters may be obtained for example, from sensors and/or from a client terminal of the user, such as a mobile device (e.g., smartphone, tablet, laptop). For example, sensors transmit data to the smartphone, and/or a GUI on the mobile device asks the user to enter additional data. The sleep-parameters may be locally computed and/or locally stored on the client terminal of the user, and/or data collected by the client terminal and/or sensors may be forwarded to a central server, where the central server computes the sleep-parameters and/or stores the sleep-parameters.

The target user may be an individual for which sleep-parameters are computed and/or obtained.

Sleep-parameters may be indicative of the user's sleep, for example, physiological data, behavioral data, temporal data, qualitative data, ambient data, and/or other types of data. Sleep-parameters may be subjective (e.g., asking a user to rate the quality of sleep from 1-10), and/or objective (e.g., number of times the user woke up during the night), as reported by user and/or obtained by a sensor and/or by an array of sensors, from data aggregating services for electronic records, and/or other sources. Sleep-parameters may include, for example, bedtime and wake up times, average heart rate throughout the night, total time spent in bed, sleep perception score, and more.

The sleep-parameters are obtained for each specific sleep session, for example, per night. Sleep-parameters may be acquired for multiple sleep sessions.

Sleep-parameters may be based on measurements and/or perceptions of the target individual indicative of perceived data. Measurements may be obtained from output of sensor(s) (e.g., physiological sensors, activity sensors) and/or may be obtained as data manually provided by the user via the physical user interface (e.g., via a GUI, gesture interface, and/or audio interface), for example, pressing an icon whenever caffeinated coffee is drunk. Perceptions of the target user may be obtained, for example, by the user manually entering data into the physical interface in response to one or more questions, for example, rate the quality of your sleep last night, how long did it take you to fall asleep, and how many times a night did you wake up. It is noted that the perceived data may be different than the measurements, for example, the user may perceive that it took 30 minutes to fall asleep, whereas a sensor may measure that it took the user 5 minutes to fall asleep.

Exemplary sleep-parameters include one or more of:
Total sleep time (e.g., measured in hours and/or minutes).
Sleep efficiency, denoting the ratio of the total time spend asleep (i.e., total sleep time) to the total amount dedicated to sleep (e.g., time spent in bed, including sleeping time and time trying to fall asleep, and/or time being awake after falling asleep).
Arousal index, denoting the total number of arousals (or short awakenings) divided by the total sleep time, and/or frequency of arousals (or short awakenings) events during the total sleep time.
Percent rapid eye movement (REM), denoting the percent of time during sleep spent in the REM state.
Percent deep sleep, denoting the percent of time during sleep spent in the deep sleep state.
Sleep satisfaction, denoting the perception of the target individual towards satisfaction from the night's sleep.
Day time sleepiness, denoting the perception of the target individual towards how tired/sleepy the user feels during awake time.
Night stress, denoting the amount of stress the target individual experiences at night.
Sleep fragmentation, denoting the number of fragmentation events experienced by the target individual during the sleeping period (e.g., the night).

Sleep-parameter may include data based on an activity performed by the target individual and/or a current mental state of the target individual during the day, which may be impacted by sleep during the night, for example:
Daytime naps. For example, the length of each nap, and time during the day of each nap.
Caffeine consumption. For example, the amount of caffeine ingested, and time during the day when each caffeine consumption occurred.
Alcohol intake. For example, the amount of alcohol ingested type of alcoholic beverage, and time during the day when each alcohol intake occurred.
Exercise. For example, the type of exercise, length of time of exercise, amount of calories burnt, and time during the day when exercise occurred.
Meals. For example, the amount of calories ingested, types of food eaten (e.g., food that give energy or foods that make one sleepy), and time during the day when each meal occurred.
Stress level. For example, an objective measure of stress (e.g., increased heart rate, increased breathing, increased perspiration) and/or a subjective measure of stress provided by the target individual. The intensity of stress, the length of time the stressful event lasts, and/or time of day of the stressful event.
Result of reaction-time game. The reaction-time game may be presented within the GUI, for example, at defined time intervals, at predefined events, and/or triggered by events, for example, after multiple activities have occurred. The reaction-time game measures a reaction time of the target individual in response to visual and/or auditory stimulus. For example, the user may be asked to press a button in the screen as fast as possible after hearing a certain sound, and/or after seeing a certain displayed image.
Perception of energy state by the target individual. For example, at wake-up, and/or at one or more times throughout the day.
Motivation. Optionally, a perception of motivation, which may be manually entered by the user via the user interface. The amount of motivation may be entered at defined time intervals and/or at trigger events.
Mood. Optionally, a perception of mood, which may be manually entered by the user via the user interface. The type of mood may be entered at defined time intervals and/or at trigger events.

One or more sleep parameters may be automatically computed according to data of one or more sensors. Exemplary sensors include physiological sensors that measure one or physiological parameters of the target individual, and/or activity sensors that measure one or more activities of the target individual.

Exemplary activity sensors include: a microphone of the client terminal (e.g. smartphone) that senses noise such as snoring or lack or noise indicating sleep or senses the voice of the target individual indicating lack of sleep, a camera of the client terminal (e.g., smartphone) that capture images (e.g., video) of the target individual sleep and code that analyzes the images to compute the sleep-parameters, a location-based sensor (e.g., GPS) that senses the geographic location of the target individual, a mobility sensor that determines whether the target user is still (i.e., in bed sleeping or trying to sleep) or walking around, a bed-movement sensor that determined whether the target individual is lying still in bed or is moving around in bed (e.g., restless sleep, tossing and turning) such as an accelerometer.

Exemplary physiological sensors include: a heart rate sensor that measures heart rate, an eye state sensor that measures whether the eye is open or closed and/or eye movements, a breathing sensor that measures breathing rates, and a brain signal sensor that measures brain signals (e.g., EEG).

The output of the activity and/or physiological sensor(s) may be analyzed to identify when the target individual is sleeping, the state of sleep (e.g., REM, deep sleep, light sleep), when the target individual is awake, whether the target individual is in bed trying to sleep, and/or whether the target individual is experiencing high levels of stress. For example, sleep or awake states may be estimated according to heart rate, breathing rate, and brain signals. Total time trying to sleep may be estimated from output of the mobility sensor.

Sensors may be wearable, incorporated into an object worn by the target individual, for example, a watch, a necklace, a chest belt, a ring, socks, pants, shoes, undergarments, a wrist band, a head band, a smart shirt, a wall mounted sensor, and a hat. Exemplary wearable sensors include: a heart rate sensor that senses heart rate, a movement sensor that tracks steps (i.e., walking, running), an activity sensor that senses fitness activities, a calorie sensor that estimates calories being burnt, a temperature sensor that senses body temperature, a perspiration sensor that senses perspiration, a pulse oximeter that senses hemoglobin oxygenation levels, a breathing sensor that senses respiratory rate, and an electrogram sensor that measures electrical activity of tissue (e.g., heart, brain, and muscle).

Sensors may be contactless sensors that do not directly contact the target individual, for example, indirectly contacting the target individual, for example, located under the mattress, located on the surface of the mattress, and located within a bag and/or purse being carried by the target individual. Such sensors may be implemented, for example, as accelerometers, location based sensor, microphone, and/or camera of a Smartphone.

Sensors may be internet of things (IoT) enabled, which may be implemented within household items, for example, within a coffee machine to transmit indications of when the target individual user is drinking coffee, code that analyzes security surveillance videos to determine the location and/or activity of the target individual within the home, a smart-TV that transmits indications of when the target individual is watching television, and a IoT enabled treadmill that transmits indications of when the target individual is exercising.

One or more sleep parameters may be computed according to perceived data entered by the user via the GUI. For example, the target user marks on a scale (e.g., from 1 to 10) an indication of sleep satisfaction, daytime sleepiness, and/or daytime stress.

As used herein, the general assumption is that people sleep at night and are awake during the day, however, the systems, methods, apparatus, and/or code instructions described herein are not necessarily limited to day and night, and are applicable to other scenarios where people are awake at night and sleep during the day (e.g., shift workers), are awake for extended periods of time that include night and day (e.g., on call physicians, soldiers, researchers in the arctic and/or Antarctica), and/or experience abnormal lengths of night and/or day (e.g., plane travelers).

At 206, one or more preprocessing processes may be performed.

Optionally, features are extracted from the sleep-parameters. The features are used to train the baseline machine learning model (e.g., as described with reference to 208) and/or are fed into the customized machine learning model (e.g., as described with reference to 212).

Features may be extracted by applying various functions, which may be, for example, hand crafted features, and/or features automatically learned by other trained machine learning models, such as encodings from trained neural networks. Features may be identified and extracted using automated feature identification and/or extraction approaches, such as brute force and/or heuristic approaches that test different combinations of features and select the most relevant ones.

Exemplary features include:
Weekly average bedtime
Weekly range of wake-up time.
Discrepancies between nightly sleep efficiency (percent of time spent asleep out of total time spent in bed) and a defined threshold (e.g., a constant threshold, or a threshold learned based on target individual's historical data).
Discrepancies between measured and perceived sleep parameters, e.g., sleep efficiency, sleep latency, time spent awake after sleep onset, etc.
Indicators of heart rate variability, such as RMSSD (root mean square of successive differences between normal heartbeats), SDNN (standard deviation of NN intervals), or others.
Weekly average of perceived daytime sleepiness.
Frequency content of nocturnal heart rate, and mathematical calculations of such values, e.g., the total power in a set frequency band, or the ratio between total power in two different frequency bands.
A sleep-parameter (e.g., raw, without further processing) may be used as a feature.

The features may be customized by being selected according to a set of characteristics of the target user. Features may be selected, for example, by a mapping dataset, set of rules, and/or other approaches (e.g., trained models) that maps characteristics to features. For example, features relevant to certain characteristics are selected, while other features not relevant to certain characteristics are not selected. For example, people living in cold climates may require body temperature as a feature (e.g., being cold at night may impact sleep), while people living in moderate climates may not require body temperature as a feature since the body temperature is assumed to be normal and therefore not impacting on sleep.

Weights may be computed for respective sleep-parameters and/or respective features, for example, according to the characteristics of the user. Weights may be set, for example, by a mapping dataset, set of rules, and/or other approaches (e.g., trained models) that compute weights based on features. For example, people that are obese may be at increased risk of sleep apnea, and features related to sleep apnea may be assigned higher weights. Older males may be at increased risk for enlarged prostate, and feature related to urinary frequency may be assigned higher weight. For people working stressful jobs, feature related to stress and/or coffee consumption may be assigned higher weights.

Exemplary characteristics may indicate the target user's sleep behavior and/or history and/or demographic parameters of the target user. Examples of the characteristics of the user include attributions, descriptors, labels, symptoms, tendencies, or other values which define the user's sleep behavior and history, history of sleep issues and/or disturbances and/or symptoms of such issues, demographic information such as age, BMI, medical conditions (other than sleep disorders, such as heart disease, diabetes), occupation, geographical location, gender and more, class or label obtained from a different classification process, etc.

An exemplary approach is now described:

A historical feature dataset that maps each respective historical sleep session to a respective set of feature extracted from sleep-parameters obtained from the respective historical sleep session may be created. The historical feature dataset may exclude features extracted from sleep-parameters of the at least one previous sleep session.

A principal component analysis (PCA) of the historical feature dataset may be performed by applying an alternating least squares (ALS) process and weighting observations with a temporal function indicating time from observation, to obtain a principal component coefficient dataset and a vector documenting percentage of total variance explained by each principal component. A weight dataset may be computed as a weighted average (or other aggregation operation) of the principal component coefficient matrix, with respect to values of the vector.

Weights of the weight dataset may be normalized within a defined range. Sign values of each weight in the weight dataset may be adjusted based on a predefined directions vector. Weights of missing features in the weight dataset may be proportionally distributed to available feature weights in the weight dataset.

The weight dataset may be used as the baseline machine learning model with weights set to initial baseline values (e.g., as described with reference to 202). When the baseline machine learning model with weights set to initial baseline values is implemented as the weight dataset, training the baseline machine learning model to obtain the customized machine learning model (e.g., as described with reference to 208) is implemented as a recent feature dataset of features extracted from sleep-parameters of the previous sleep session(s). The sleep wellness score is obtained as the outcome of the customized machine learning model (e.g., as described with reference to 214) by computing the sleep wellness score as a weighted sum of the recent feature dataset, with respect to the weight dataset.

At 208, the baseline machine learning model is trained using the sleep-parameters of the historical sleep sessions of the target individual. The baseline machine learning model may be trained by adjusting the initial baseline values of the weights, to obtain a customized machine learning model. During iterations, the previous baseline values of the weights may be adjusted to obtain updated current values of the current customized machine learning model.

Optionally, the adjustment of the weights is performed based on the historical weights in view of currently computed weights, for example, by normalizing the weights in view of the currently computed weights, and/or computing an average (e.g., weighted average) of the historical and currently computed weights.

The baseline machine learning model and/or the customized machine learning model may be implemented as an auto-regressive model. The auto-regressive model may, for example, adjust (e.g., increase and/or decrease) weights where the current weight is statistically significantly different than a trend in historical weights, and/or may maintain the weights where the current weight is statistically similar to a trend in historical weights.

Other exemplary architectures of the baseline machine learning model and/or the customized machine learning model include, for example, statistical classifiers and/or other statistical models, neural networks of various architectures (e.g., convolutional, fully connected, deep, encoder-decoder, recurrent, graph), support vector machines (SVM), logistic regression, k-nearest neighbor, decision trees, boosting, random forest, a regressor, and/or any other commercial or open source package allowing regression, classification, dimensional reduction, supervised, unsupervised, semi-supervised or reinforcement learning. Machine learning models may be trained using supervised approaches and/or unsupervised approaches.

The same baseline machine learning model may be used for multiple individuals. The same baseline machine learning model may then be trained to generate the customized machine learning model, which is specific for each individual.

During iterations, the baseline machine learning model may be further trained using sleep wellness scores previously obtained by earlier versions of the customized machine learning model, to obtain the current customized machine learning model.

At 210, sleep-parameters computed for one or more previous sleep session of the target individual are accessed, for example, obtained from sensors, from a computing device, from a server, and/or from a data storage device, for example, as described with reference to 204. The sleep-parameters obtained for the one or more previous sleep sessions are obtained during previous sleep sessions that are after the historical sleep sessions' sleep-parameters (e.g., as in 204) used to train the baseline machine learning model (e.g., as in 208). I.e., the sleep-parameters obtained for the previous sleep session(s) are not used to train the baseline machine learning model.

At 212, the sleep-parameters and/or features and/or other data computed for the one or more previous sleep session are fed into the customized machine learning model.

Data fed into the model may be from a single previous sleep session, and/or from multiple previous sleep sessions. Data from multiple previous sleep sessions may be arranged in a sequence and/or labelled with a date, indicating relative arrangement of the data according to previous nights. Data of more recent nights may be assigned higher weights relative to data from earlier nights, for example, indicating that the data of more recent nights is more relevant. For example, recent habits (e.g., recent coffee consumption, sleep schedule) reflect the current reality more than older habits which may not be relevant.

Optionally, after a previous iteration where a sleep wellness score has been obtained (e.g. as described with reference to 214), the previously computed historical sleep wellness score(s) may be fed as input into the current version of the customized machine learning model along with the respective sleep-parameter for the most recent previous sleep session. For example, as a single vector that simultaneously includes the previously computed historical sleep wellness score(s) and the sleep-parameter.

At 214, a sleep wellness score is obtained as an outcome of the customized machine learning model. The sleep wellness score may be represented, for example, as an absolute numerical value (e.g., on a scale of 0-100), a relative value (e.g., 0-1). The numerical value of the sleep wellness score may be into one of multiple classification categories based on predefined thresholds. The threshold may be predefined or determined by a different process, for example, learned by another machine learning model. Alternatively or additionally, the sleep wellness score is directly obtained as the classification category as the outcome of the customized machine learning model, for example, low, medium, and high.

The sleep wellness score may be locally computed by the client terminal of the user, and/or centrally computed by the central server which may provide the sleep wellness score to the client terminal of the user. Other related features described herein (e.g., analyze the sleep wellness score for a single sleep session and/or for multiple sleep sessions, identifying the sleep condition, generating instructions, classification of the sleep wellness score, and identifying the most significant sleep-parameters impacting the sleep wellness score) may be locally computed by the client terminal and/or centrally computed by the server and provided to respective client terminals.

At 216, one or more post processing processes may be performed, for example, one or more of the following.

The sleep wellness score may be heuristically corrected based on predefined discrepancies between the sleep wellness score and values of predefined features.

One or more features having largest negative or positive effect on the sleep wellness score may be determined, for example, by the following process. The features of the historical feature dataset may be grouped into feature groups. A weighted contribution of respective features of each respective feature group on the sleep wellness score may be computed based on the weight dataset and the recent feature dataset. One or more feature groups which had a largest negative or positive contribution to the sleep wellness score with absolute values larger than a threshold, may be selected. It is noted that it is possible that no feature groups is selected by the described process.

A reliability level for the calculation of the sleep wellness score may be computed, for example, based on one or more of: (i) an amount of data missing from the recent feature dataset, (ii) a number of features in the historical feature dataset, and (iii) amount of data missing from the historical feature dataset.

The sleep wellness score may be analyzed, optionally in view of a historical trend of previously computed sleep wellness scores, to determine whether the current sleep wellness score is an anomaly with respective to the historical trend (e.g., statistically significantly higher or lower than a historical baseline defined by the trend), or normal with respect to the historical trend (e.g., within a statistical distribution of the historical baseline defined by the trend). Anomalies may be flagged and/or further analyzed, for example, to determine whether they represent a positive improvement to the sleep condition and/or negative effect on the sleep condition, and/or sleep-parameters contributing to the anomaly. Instructions for treatment may be generated according to the anomaly, for example, how to “fix” the sleep-parameters to improve the sleep wellness score.

At 218, a sleep condition may be identified by analyzing the sleep wellness score. For example, the sleep wellness score may be fed into an application that generates the sleep condition as an outcome. Examples of applications include, a sleep evaluation application, a sleep improvement application, a sleep monitoring application, and a sleep maintenance application.

The application may be selected, for example, as a default setting, by the user, and/or based on a set of rules.

At 220, the sleep condition and/or the sleep wellness score may be provided. For example, presented on a display, stored on a data storage device, forwarded to another device (e.g., remote server), and/or fed into another process.

Optionally, instructions for treating the target user for the sleep condition and/or for gaining insights into the sleep condition are presented on a display and/or for playing on speakers. The instructions may be obtained, for example, by a mapping dataset that maps the sleep condition and/or the sleep wellness score to predefined instructions, and/or by an instruction machine learning model that is trained on a training dataset obtained for multiple sample individuals that includes sleep conditions and/or sleep wellness scores, corresponding instructions (e.g., defined by a domain expert such as a sleep expert), and optionally feedback (e.g., from the users, from a computing device and/or server) indicating the effect of the instructions on the sleep condition and/or sleep wellness scores. In this manner, the instructions most likely to improve the sleep condition and/or the sleep wellness scores may be provided.

Examples of instructions to improve the sleep condition and/or the sleep wellness scores include:
Get more hours of sleep.
Reduce caffeine intake throughout the day.
Reduce or stop fluid intake 3 hours before going to bed.
Change sleep schedule, for example, go to sleep at 10 PM rather than midnight, and wake up at 6 AM.
Exercise during the day, but not during the 3 hours before going to bed.
Change pillow and/or mattress to increase comfort.
Use earplugs to reduce noise.

The sleep condition and/or the sleep wellness score and/or the instructions may be for other cases (i.e., not necessarily for a sleep disorder that is to be improved), for example:
For a target individual who is a healthy sleeper and wish to gain additional insights into her sleep.
For a target individual who is not suffering from a sleep disorder but does experience some sleep difficulties.
For a target individual who is suffering from a sleep disorder.
For a target individual who suffered from a sleep disorder or did not suffer from a sleep disorder but did experience some sleep difficulties in the past, and is now maintaining healthy sleep, as part of a relapse prevention program.
As part of a sleep evaluation program.
As part of a sleep improvement program, for a target individual who suffers from sleep disorders.
As part of a sleep improvement program, for a target individual who does not suffer from sleep disorders but does experience some sleep difficulties.
As part of a sleep maintenance and/or relapse prevention program.
As a tool for target individuals who are healthy sleepers and wish to gain insights into their sleep.

In an example, a user is classified by a different application as suffering from symptoms of insomnia. User's sleep-parameter are then assessed as described herein on a nightly basis, as each night receives a sleep wellness score and/or a category, with a list of sleep-parameters impairing each score. For each night, the sleep wellness score may be classified as anomalous or normal. The outcomes of the process described herein (e.g., sleep wellness scores, categories, impairing factors, anomaly classification label) may be used by different systems (e.g., application), for example, as part of a sleep improvement program, a sleep maintenance program, etc. If, for example, a low sleep wellness score is detected, a sleep maintenance program may use this information and suggest treatment, tools, techniques, and more, which will assist user in regaining higher quality of sleep.

At 222 one or more features described with reference to 202-220 may be iterated, for example, over one or more sleep sessions over multiple sequential time intervals, for example, per day, per 3 days, per week, and the like.

During iterations, the baseline machine learning model (e.g., described with reference to 202) is implemented as a previous version of the customized machine learning model previously trained on sleep-parameters for historical sleep session. The customized machine learning model (e.g. as described with reference to 208) is a current version thereof trained on sleep-parameters of most recent sleep session that is later than the historical sleep session.

During the iterations over sequential time intervals, the current version of the customized machine learning model is dynamically re-trained (e.g., as described with reference to 208) using the sleep-parameters for most recent historical sleep session, optionally by adjusting previously computed values of the weights of previous versions of the customized machine learning model. Sleep-parameter for the most recent previous sleep session are accessed (e.g., as described with reference to 210), and fed into the current version of the customized machine learning model (e.g., as described with reference to 212) for obtaining a current sleep wellness score for each most recent previous sleep session (e.g., as described with reference to 214).

During the iterations over sequential time intervals, the customized machine learning model may be retrained (e.g., as described with reference to 208) using the sleep-parameters for the most recent historical sleep session labelled with the sleep wellness score obtained as an outcome of the previous version of the customized machine learning model.

Optionally, (e.g., during the post processing as in 216) multiple sleep wellness scores obtained over multiple sequential time intervals are analyzed to detect a statistically significant deviation of one or more certain sleep wellness scores, for example, a night where a sleep wellness score was very high and/or very low compared to the other nights. Sleep-parameter(s) most significantly contributing to the statistically significant deviated certain sleep wellness score(s) outcome by the customized machine learning model may be computed, for example, using machine learning interpretability approaches. The identified sleep-parameters may be presented on the display. The instructions (e.g. as in 220) may relate to the identified sleep-parameters, for example, pointing out to the user which sleep-parameters were important to obtain a good night sleep according to the statistically significant change in sleep wellness score indicating a good night's sleep, and/or instructions to maintain and/or reproduce the sleep-parameters in the future to obtain additional nights with good sleep. In another example, the instructions may point out to the user which sleep-parameters were the cause of the poor night sleep according to the statistically significant change in sleep wellness score indicating the poor night's sleep, and/or the instructions may be for how to change the sleep-parameter to reduce or avoid future poor sleep.

Optionally, weights of the sleep-parameters are analyzed and iteratively adjusted over multiple sleep sessions. The adjusted weighted sleep-parameters may be the ones used to train the current version of the customized machine learning model, and/or inputted into the current version of the customized machine learning model. Exemplary adjustments include: (i) reducing previously computed values of a first sub-set of the weights associated with a first sub-set of sleep-parameters that are statistically constant over multiple sleep sessions. Sleep-parameters that are constant may not significantly impact the sleep wellness score and/or may not be subject to change, for example, representing strong habits of the user that cannot be changed, such as a user that works shift from 11 PM to 7 AM which cannot be changed, and/or a user that is used to drinking 6 cups of coffee a day and cannot function without them. (ii) Increasing previously computed values of a second sub-set of the weights associated with a second sub-set of sleep-parameters that are statistically varying over the sleep sessions. Sleep-parameters that change may significantly impact the sleep wellness score and/or may be subject to change, for example, representing flexible habits of the user that can be changed, for example, different foods that the user eats, and/or exercise.

Referring now back to FIG. 3, at 302, the process starts.

At 304, when the sleep-parameters are available for the most recent sleep session (e.g., recent night) the process continues. Else, the process waits for the sleep-parameters of the most recent sleep session to be available.

At 306, when the user recorded (i.e., obtained sleep-parameters for) at least N_{nights} nights in the past N_{days} days, the process continues. Else, wait for user to comply with this condition.

At 308, the set of features, denoted F, which will be used for user, based on user's characteristics matrix, denoted UC, is determined.

F in a matrix, as each row corresponds to a single night recording and each column corresponds to a single feature, denoted F(i). Matrix F contains data for NF nights, not including the data of the most recent night.

At 310, columns of F, are normalized to get a normalized matrix, denoted Fₙ.

At 312, when a minimal number of columns, denoted (N_{features}), of Fₙ comply with the following conditions, the process continues:
The number of missing values in the column Fₙ(i) does not exceed a predefined threshold.
The number of values in the column Fₙ(i) smaller than a predefined threshold, does not exceed a predefined threshold.
The variability of values in the column Fₙ(i) is not smaller than a predefined threshold.

Else, the process ends without calculating the Sleep Wellness Score:

At 314, When a Sleep Wellness Score was calculated for the user in a previous interval of time, with a length of such time predefined, continue to 316. Else, go to 340.

At 316, a vector, denoted x, containing the set of features defined for F, is generated based on the sleep data (i.e., the sleep-parameters) of the recent sleep session.

At 318, x is normalized by using M and S which were calculated for generating Fₙ, to get xₙ.

At 320, a Principal Component Analysis (PCA) of the matrix Fₙ is performed by applying the Alternating Least Squares (ALS) algorithm and weighting observations in Fₙ with a temporal function ƒ_{w}(t), where t denotes time from observation. ƒ_{w}(t) can be, e.g., a reciprocal function.

At 322, using the principal component coefficients matrix C and the vector Sc containing the percentage of the total variance explained by each principal component, the weights vector, denoted w, is computed as the weighted average of columns of C, with respect to the values of Sc.

At 324, w is processed by: normalizing w to the range [−1,1], adjusting the sign of each value in w based on a predefined directions vector, denoted d, and proportionally distributing weights of missing features in w to all available features weights in w.

At 326, the normalized Sleep Wellness Score value, denoted SWSₙ, is calculated as the weighted sum of x with respect to w, i.e., SWSₙ=sum(w*xₙ).

At 328, SWSₙ is scaled to the range [min_score,max_score], based on characteristics of previous SWSₙ values. For example, [min_score,max_score] can be the range [0,100]. The scaled value is the Sleep Wellness Score, or SWS.

At 330, values of SWS are heuristically corrected based on predefined discrepancies between SWS value and the values of predefined features.

At 332, a determination is made whether the value of SWS is anomalous, based on previous values of SWS. Such anomaly detection can be computed heuristically or, e.g., with an algorithm which is statistical distribution-based, distance-based, density-based, deviation-based, regression-based, or other outlier detection algorithms.

At 334, the Top Impairing Factors are determined as the set of features which had the largest negative effect on SWS, by the following process: features used in F and x are grouped in features groups, denoted G. The weighted contribution of the features in each group G(i), on SWS, are calculated based on w(i)*x(i). The top N_{TIF} groups of G which had the largest negative contribution on SWS, with absolute values larger than a predefined threshold, is selected.

At 336, the value of SWS may be classified into one of several score classes, or categories, based on predefined threshold. Classes, or categories, for example, “Low”, “Medium”, “High”, color codes, etc.

At 338, the reliability level for the calculation of SWS, based on the amount of missing data in x, the number of features used in F, and/or the amount of missing data in F, is calculated.

Referring now back to 340, SWS is computed for previous sleep sessions of the user, including the most recent sleep session, based on general specifications for a single night, where features 320B-338B are performed by adapting features described with reference to 320A-338A, where it is noted that there are no features necessarily corresponding to 332A-334A, by performing calculations by considering matrix Fₙ instead of vector xₙ. In particular:
At 322B, a weights matrix, denoted W, is calculated from the Principal Components Analysis of matrix Fₙ.
At 326B, a normalized Sleep Wellness Scores vector, denoted SWSₙ, is calculated as the weighted sum of F with respect to W.
At 328B, SWSₙ is scaled to the range [min_score,max_score], to get Sleep Wellness Score vector SWS.
At 330B, the values of SWS are heuristically corrected based on predefined discrepancies between SWS values and the values of predefined features.
At 336B, each value of the SWS vector is classified to a class or a category.
At 338B, the reliability level for the calculation of each value of SWS is calculated, while also considering the number of sleep sessions used in F.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant machine learning models will be developed and the scope of the term machine learning model is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A computer implemented method of training a machine learning model for generating a sleep wellness score used for treatment of a sleep condition in a target individual, comprising:
providing a baseline machine learning model with a plurality of weights set to initial baseline values;
accessing a plurality of sleep-parameters computed for a plurality of historical sleep sessions of the target individual;
training the baseline machine learning model using the plurality of sleep-parameters for the plurality of historical sleep sessions of the target individual by adjusting the initial baseline values of the plurality of weights, to obtain a customized machine learning model;
accessing a plurality of sleep-parameters computed for at least one previous sleep session of the target individual;
inputting the plurality of sleep-parameters computed for at least one previous sleep session into the customized machine learning model; and
obtaining a sleep wellness score as an outcome of the customized machine learning model.

2. The method of claim 1, further comprising analyzing the sleep wellness score to identify the sleep condition by feeding the sleep wellness score into an application selected from a group consisting of: a sleep evaluation application, a sleep improvement application, a sleep monitoring application, a sleep maintenance application;
and presenting instructions on a display and/or for playing on speakers for treating the target user for the sleep condition and/or for gaining insights into the sleep condition according to the analysis.

3. The method of claim 1,
wherein the baseline machine learning model comprises a previous version of the customized machine learning model previously trained on sleep-parameters for historical sleep session, and the customized machine learning model comprises a current version thereof trained on sleep-parameters of most recent sleep session that is later than the historical sleep session; and
further comprising:
iterating over a plurality of sequential time intervals, dynamically re-training the current version of the customized machine learning model using the plurality of sleep-parameters for most recent historical sleep session by adjusting previously computed values of the plurality of weights of previous versions of the customized machine learning model,
wherein the accessing comprises accessing the plurality of sleep-parameter for the most recent previous sleep session,
and iterating the inputting, and the obtaining over the plurality of sequential time intervals to obtain a respective sleep wellness score for each most recent previous sleep session.

4. The method of claim 3, wherein dynamically re-training comprises re-training the customized machine learning model using the plurality of sleep-parameters for the most recent historical sleep session labelled with the sleep wellness score obtained as an outcome of the previous version of the customized machine learning model,
wherein a current version of the customized machine learning model is further fed an input of at least one historical sleep wellness score with respective plurality of sleep-parameter for the most recent previous sleep session.

5. The method of claim 3, further comprising analyzing a plurality of sleep wellness scores obtained over the plurality of sequential time intervals to detect a statistically significant deviation of a certain sleep wellness score, and identifying at least one sleep-parameter most significantly contributing to the certain sleep wellness score outcome by the customized machine learning model.

6. The method of claim 3, further comprising:
at least one of: (i) reducing previously computed values of a first sub-set of the plurality of weights associated with a first sub-set of sleep-parameters that are statistically constant over a plurality of sleep sessions, and (ii) increasing previously computed values of a second sub-set of the plurality of weights associated with a second sub-set of sleep-parameters that are statistically varying over the plurality of sleep sessions.

7. The method of claim 1, wherein the baseline machine learning model and the customized machine learning model are implemented as an auto-regressive model.

8. The method of claim 1, wherein the initial baseline values of the plurality of weights are initially set to random values.

9. The method of claim 1, wherein the baseline machine learning model is trained on a training dataset that includes a plurality of sample sleep-parameters labelled with respective sample sleep wellness scores denoting ground truth for a plurality of sample individuals, wherein the training the baseline machine learning model to obtain the customized machine learning model is done on a customized training dataset that includes the plurality of sleep-parameters of the target individual and excludes sleep-parameters of other individuals.

10. The method of claim 1, further comprising extracting a plurality of features from the plurality of sleep-parameters, wherein the plurality of features are used to train the baseline machine learning model and/or are fed into the customized machine learning model.

11. The method of claim 10, wherein the plurality of features are customized by being selected according to a set of characteristics of the target user denoting the target user's sleep behavior and/or history and/or demographic parameters of the target user.

12. The method of claim 10, further comprising creating a historical feature dataset that maps each respective historical sleep session to a respective set of feature extracted from sleep-parameters obtained from the respective historical sleep session, wherein the historical feature dataset excludes features extracted from sleep-parameters of the at least one previous sleep session.

13. The method of claim 12, further comprising:
performing a principal component analysis (PCA) of the historical feature dataset by applying an alternating least squares (ALS) process and weighting observations with a temporal function indicating time from observation, to obtain a principal component coefficient dataset and a vector documenting percentage of total variance explained by each principal component;
computing a weight dataset as a weighted average of the principal component coefficient matrix, with respect to values of the vector,
wherein baseline machine learning model with weights set to initial baseline values is implemented as the weight dataset.

14. The method of claim 13, further comprising:
normalize weights of the weight dataset within a defined range;
adjust sign values of each weight in the weight dataset based on a predefined directions vector; and
proportionally distribute weights of missing features in the weight dataset to available feature weights in the weight dataset.

15. The method of claim 14, wherein baseline machine learning model with weights set to initial baseline values is implemented as the weight dataset,
wherein training the baseline machine learning model to obtain the customized machine learning model comprises a recent feature dataset of features extracted from sleep-parameters of the at least one previous sleep session,
wherein obtaining the sleep wellness score as the outcome of the customized machine learning model comprises computing the sleep wellness score as a weighted sum of the recent feature dataset, with respect to the weight dataset.

16. The method of claim 15, further comprising heuristically correcting the sleep wellness score based on predefined discrepancies between the sleep wellness score and values of predefined features.

17. The method of claim 15, further comprising computing at least one feature having largest negative effect on the sleep wellness score, by:
grouping the features of the historical feature dataset into a plurality of feature groups;
computing a weighted contribution of respective features of each respective feature group on the sleep wellness score based on the weight dataset and the recent feature dataset;
selecting at least one feature group which had a largest negative contribution to the sleep wellness score with absolute values larger than a threshold.

18. The method of claim 15, wherein the sleep wellness score is represented as a numerical value, and further comprising classifying the sleep wellness score into one of a plurality of classification categories based on predefined thresholds.

19. The method of claim 15, further comprising computing a reliability level for the calculation of the sleep wellness score based on at least one of: (i) an amount of data missing from the recent feature dataset, (ii) a number of features in the historical feature dataset, and (iii) amount of data missing from the historical feature dataset.

20. A computer implemented method of using a machine learning model for generating a sleep wellness score used for treatment of a sleep condition in a target individual, comprising:
accessing a plurality of sleep-parameters computed for at least one previous sleep session of the target individual;
inputting the plurality of sleep-parameters computed for at least one previous sleep session into a customized machine learning model; and
obtaining a sleep wellness score as an outcome of the customized machine learning model,
wherein the customized machine learning model is trained by:
providing a baseline machine learning model with a plurality of weights set to initial baseline values,
accessing a plurality of sleep-parameters computed for a plurality of historical sleep sessions of the target individual, and
training the baseline machine learning model using the plurality of sleep-parameters for the plurality of historical sleep sessions of the target individual by adjusting the initial baseline values of the plurality of weights, to obtain a customized machine learning model.

21. A system for training a machine learning model for generating a sleep wellness score used for treatment of a sleep condition in a target individual, comprising:
at least one hardware processing executing a code for:
accessing a baseline machine learning model with a plurality of weights set to initial baseline values;
accessing a plurality of sleep-parameters computed for a plurality of historical sleep sessions of the target individual;
training the baseline machine learning model using the plurality of sleep-parameters for the plurality of historical sleep sessions of the target individual by adjusting the initial baseline values of the plurality of weights, to obtain a customized machine learning model;
accessing a plurality of sleep-parameters computed for at least one previous sleep session of the target individual;
inputting the plurality of sleep-parameters computed for at least one previous sleep session into the customized machine learning model; and
obtaining a sleep wellness score as an outcome of the customized machine learning model.
